(19) United States
(12) Patent Application Publication
(10) Pub. No.: US 2022/0120724 A1
(43) Pub. Date: Apr. 21, 2022

(54) DETECTOR

(71) Applicant: Total Waste Solutions Ltd, Sheffield (GB)

(72) Inventors: Paul Sharman, Sheffield (GB); Graham Bland, Sheffield (GB); Timothy Hale, Sheffield (GB)

(21) Appl. No.: 17/310,594

(22) PCT Filed: Feb. 12, 2020
(86) PCT No.: PCT/GB2020/050327

§ 371 Date: Aug. 12, 2021

(30) Foreign Application Priority Data
Feb. 13, 2019 (GB) 1902004.9

(51) Int. Cl. G01N 33/00; B65F 1/14; G01N 33/497

(52) U.S. Cl. CPC G01N 33/0063; B65F 1/14; B65F 2001/1692; G01N 33/0031; B65F 2210/168; G01N 33/497

(57) ABSTRACT

A detector (10; 110) is provided for mounting to a container (2) defining an internal space (6) in order to determine occupancy of the container. The detector includes a housing (12; 112), a mounting arrangement for mounting the housing to a container, a sensor arrangement comprising a first sensor (30; 130) disposed within the housing, the first sensor configured to determine a concentration of carbon dioxide within an internal space (6) of a container (2); and a control system (40; 140) configured to receive input values from the first sensor and to provide an output when the concentration of carbon dioxide exceeds a predetermined value. The housing is substantially sealed for preventing the ingress of debris into the housing, and the housing is configured such that air is able to flow into the housing and over the first sensor.

### FIELD OF THE INVENTION

The present invention relates to a detector for determining the occupancy of a container, and to a container including detector for determining the occupancy thereof.

### BACKGROUND OF THE INVENTION

In order to find shelter from harsh weather conditions, it is becoming an increasingly common occurrence for people to find rest in a refuse or recycling container (also known as a bin). It is possible for occupants of such a container to be severely injured, e.g. if they are transferred from the container to a refuse/recycling compacting collection vehicle. In light of this, there is growing concern within the refuse/recycling collection industry about the rising number of people sleeping in such containers.

In order to reduce the risk of an occupant of a container becoming injured, it is common practice for operators (i.e. refuse/recycling collectors) to try and determine the occupancy of a container prior to emptying the recycling/refuse in the container to a collection vehicle. Traditional methods require the operator to investigate the internal space of the container, e.g. by moving waste around, shouting, making noises on the side of the bin etc. However, these methods are not always effective, e.g. if a person within a container does not wake up, or is not visible to an operator.

The present invention seeks to overcome or at least mitigate one or more problems associated with the prior art.

### SUMMARY OF THE INVENTION

According to a first aspect there is provided a detector for mounting to a container defining an internal space to determine occupancy of the container, the detector comprising: a housing defining an internal volume; a mounting arrangement for mounting the housing to a container; a sensor arrangement comprising a first sensor disposed within the housing, the first sensor configured to determine a concentration of carbon dioxide within an internal space of a container; and a control system configured to receive input values from the first sensor and to provide an output when the concentration of carbon dioxide exceeds a predetermined value, wherein the housing is substantially sealed for restricting the ingress of debris into the housing, and wherein the housing is configured such that air is able to flow into the housing and over the first sensor.

People are a natural emitter of carbon dioxide through respiration. When people take shelter within an enclosed container for a period of time, the concentration of carbon dioxide within the air in the container rises. The present arrangement provides a detector that is able to be mounted within a range of different containers so as to be able to monitor the levels of carbon dioxide therein. This allows for existing containers to be retro-fitted with a detector.

The housing is substantially sealed and has an inlet that is configured so as to prevent dust ingress into the housing, whilst enabling air to flow therein. This arrangement enabled the detector to function, whilst preventing dirt, waste and water (e.g. rain) from entering the housing to interfere with the detector. Providing a housing that is sealed (e.g. water-tight and/or dust-tight), whilst allowing air to flow into the housing, minimises ingress of water and/or debris into the housing that could damage the internal components of the detector.

The detector may be configured to be mounted to an external surface of a container, in use, and wherein the housing may comprise a sensor housing portion defining an inlet for air to flow into the sensor housing portion, the sensor housing portion configured and arranged to extend into a container, in use.

Providing a sensor housing portion that projects from the housing into a container enables the detector to be positioned on an external surface of a container, which ensures the detector is highly visible to an operator approaching the container.

The detector may be configured to be mounted to an external surface of a container, in use, and wherein the housing may comprise a sensor housing portion defining an inlet for air to flow into the sensor housing portion, the sensor housing portion extending from the housing and being configured and arranged to extend through an opening of a wall of a container, in use.

Providing a sensor housing portion that projects from the housing enables it to extend through an opening the container wall. This enables the detector to be positioned on an external surface of a container, which ensures the detector is highly visible to an operator approaching the container.

By extending through a wall of the container, as opposed to extending over an upper edge of a container wall, for example, enables a more compact detector to be produced.

Positioning the detector away from the upper edge of a container wall also helps to avoid damage to the detector from impacts, e.g. from a container lid.

The inlet may have a diameter in the range of 1 mm to 3 mm, for example the diameter of the inlet may be approximately 2 mm.

Providing apertures within this range of sizes enables air to pass into the housing and over the sensor arrangement, whilst inhibiting debris and/or water from entering into the housing.

The housing may comprise a cover substantially covering the inlet, and wherein the cover may be provided as a semi-permeable membrane configured to allow the passage of air therethrough and configured to prevent the passage of water therethrough.

An inlet is provided to allow air to flow into the housing, and so to flow over the first sensor. Provision of the cover minimises ingress of debris into the housing via the inlet which could damage the internal components of the detector. The use of a semi-permeable membrane provides an effective means of preventing water ingress whilst allowing air to flow into the housing.

The mounting arrangement may comprise at least one fastener configured and arranged to extend through an outer wall of a container, in use, in order to mount the detector to a container.

The mounting arrangement may comprise a mounting plate for positioning within a container such that a section of a container is positioned between the mounting plate and the housing, in use.

The mounting plate provides a more secure mounting of the detector to the container.

The mounting plate may comprise an angled upper region, wherein said angled upper region extends in a direction towards the housing, in use.

The upper region may be placed directly above the inlet on the sensor housing portion such that the projection and the aperture are shielded from being damaged by waste falling into the container.

The mounting arrangement may comprise a dampening arrangement for dampening vibrations between the detector and a container, in use.

The dampening arrangement may comprise a first dampening member for positioning between the housing and the container, in use.

Mounting the detector a container via one or more dampeners reduces the shock/impact from the container to the detector, e.g. during movement/loading/emptying of the container. This reduces the impact damage imparted on the components of the detector, thus increasing the service life of the detector.

Providing a dampening member (e.g. a shock absorbing washer) between the housing and the container has been found to effectively dampen the shock/impact/vibration between the container and the detector, e.g. during movement/loading/emptying of the container. This reduces the impact reduces damage imparted on the components of the detector.

The detector may comprise a first indicator, and wherein the output of the control system changes the first indicator from an inactive state to an active state for alerting an operator.

The indicator may comprise an audible indicator and/or a visual indicator.

The detector may be configured to change the first indicator from an active state to an inactive state during unloading of a container, in use.

This arrangement enables the detector, i.e. via the control system, to automatically reset the indicator during the process an emptying the container (i.e. during a discharge of the contents of the container) without the need for operator interference.

The sensor arrangement may comprise a sensor configured to determine an orientation of a container, in use, and wherein the control system may be configured to change the first indicator from an active state to an inactive state when the container has been rotated by at least a predetermined angle, optionally wherein the predetermined angle is approximately 90 degrees.

The provision of a sensor, e.g. a gyroscope, allows the detector to determine when the container has been emptied, e.g. into a larger container of a waste lorry, thus enabling it to reset the indicator from an active state to an inactive state automatically.

The sensor arrangement may be configured to detect the temperature within a container, and wherein the control system may be configured to receive input values from the sensor arrangement and to provide an output when the temperature within the container exceeds a pre-determined value.

Monitoring the temperature within a container further aids in the detection of occupancy of the container. Should the temperature within a container rise above a predetermined value, this may be caused by an occupant within the container, and so the control system alerts an operator.

The sensor arrangement may be configured to detect temperature outside of a container, and wherein the control system may be configured to receive input values from the sensor arrangement and to provide an output when the difference between the temperature inside a container and the temperature outside of a container exceeds a pre-determined value.

Monitoring the relative temperature inside and outside of the container increases the reliability of the determination of the occupancy of the container. This comparative temperature monitoring enables the control system to be used in different climates, or over different seasons, without requiring any reconfiguration.

The sensor arrangement may be configured to detect motion within a container, and wherein the control system may be configured to receive input values from the sensor arrangement and to provide an output when motion within a container is detected, in use.

Monitoring movement within the container further increases the reliability of the determination of the occupancy of the container.

The sensor arrangement may be configured to monitor humidity within a container, and wherein the control system may be configured to receive input values from the sensor arrangement and to provide an output when the humidity within a container exceeds a predetermined value.

Monitoring humidity within the container further increases the reliability of the determination of the occupancy of the container, as the raised humidity may be the result of an occupant's breathing.

The first sensor may be configured to determine a concentration of volatile organic compounds (VOCs) within the internal space of the container, and optionally, wherein the control system may be configured to provide an output when the concentration of VOCs exceeds a predetermined value.

Monitoring the concentration of a range of VOCs within a container has been found to further increase the reliability of the determination of the occupancy of a container.

The control system may be configured to determine a mean value from the input values received from the sensor arrangement, and wherein the control system may be configured to provide an output when an input value differs from its associated mean value by a predetermined amount.

This allows the control system to learn the average sensor readings for a particular container in a particular location. Significant perturbations from the average sensor readings may help to determine that a human is within the container.

The detector may comprise a fire suppressant device disposed within the housing, and wherein the fire suppressant device is configured to activate in response to detection of fire within the housing.

The detector may comprise a power storage unit disposed within the housing for providing power to the sensor arrangement and the alarm, wherein the power storage is mounted within the housing via an anti-vibration mounting arrangement.

The predetermined concentration of carbon dioxide may be in the range of 1000 ppm to 1500 ppm, preferably in the range of 1100 ppm to 1300 ppm, for example approximately 1200 ppm.

The sensor arrangement may be configured to monitor the container at least every minute, optionally every 30 seconds, for example every 10 seconds.

The detector may comprise a transmitter for transmitting a signal indicative of the occupancy of a container to a processor at a remote location when the concentration of carbon dioxide sensed by the first sensor exceeds the predetermined value, and wherein the control system output is configured to activate the transmitter.

According to a second aspect there is provided a detector for mounting to a container defining an internal space to determine occupancy of the container, the detector comprising: a housing defining an internal volume, the housing being substantially sealed for restricting the ingress of debris into the housing; a mounting arrangement for mounting the housing to a container; a sensor arrangement comprising a first sensor disposed within the housing, the first sensor configured to monitor conditions within an internal space of a container, wherein the sensor arrangement is configured to monitor one or more of: the concentration of carbon dioxide within the container; the temperature within a container or a temperature differential between inside and outside of a container; motion within a container; concentration of VOCs within a container; and/or when a heartbeat of an occupant within a container; and a control system configured to receive input values from the first sensor and to provide an output when: the concentration of carbon dioxide exceeds a predetermined value; the temperature within a container exceeds a predetermined value or the temperature differential between inside and outside of the container exceeds a predetermine value; motion is detected within a container; concentration of VOCs exceeds a predetermined value; and/or when a cardioballistic detector detects a heartbeat within a container; wherein the housing is configured such that air is able to flow into the housing and over the first sensor.

According to a third aspect, there is provided a detector for mounting to a container defining an internal space to determine occupancy of the container, the detector comprising: a housing for positioning within a container; a mounting arrangement for mounting the housing to a container; a sensor arrangement comprising a first sensor disposed within the housing, the first sensor configured to determine a concentration of carbon dioxide within an internal space of a container; and a control system configured to receive input values from the first sensor and to provide an output when the concentration of carbon dioxide exceeds a predetermined value, wherein the housing is substantially sealed for preventing the ingress of debris into the housing, and wherein the housing is configured such that air is able to flow into the housing and over the first sensor.

The housing may comprise an inlet for allowing air to flow into the housing and an inlet cover substantially covering the inlet, wherein the inlet cover may be provided as a semi-permeable membrane configured to allow the passage of air therethrough and configured to prevent the passage of water therethrough.

An inlet is provided to allow air to flow into the housing, and so to flow over the first sensor. Provision of the cover minimises ingress of debris into the housing via the inlet which could damage the internal components of the detector. The use of a semi-permeable membrane provides an effective means of preventing water ingress whilst allowing air to flow into the housing.

The housing may comprise an outlet such that the housing defines an air flow path through the housing, and wherein the first sensor is disposed along the air flow path.

Providing an airflow path through the housing (e.g. in the form of an enclosed duct) has been found to result increased air flow through the housing and over the first sensor, which increases the accuracy of the sensing of the composition of the air within a container. Providing the air flow path in the form of a duct further increases air flow over the sensor via convection effects.

The inlet may be provided on a surface of the housing intended to be lowermost in use, and wherein the outlet may be provided on a surface of the housing that is intended to be uppermost in use.

This arrangement has been further found to further increases air flow over the sensor via convection effects.

The housing may comprise an outlet cover substantially covering the outlet. The outlet cover may be provided as a semi-permeable membrane configured to allow the passage of air therethrough and configured to prevent the passage of water therethrough.

Provision of the cover minimises ingress of debris into the housing via the inlet which could damage the internal components of the detector. The use of a semi-permeable membrane provides an effective means of preventing water ingress whilst allowing air to flow into the housing.

The housing inlet and/or housing outlet may define a diameter in the range of 10 mm to 14 mm, preferable approximately 12 mm.

Providing the inlet and/or outlet with a diameter in this range has been found to provide sufficient air flow while minimising ingress of foreign objects and preventing tampering with the first sensor.

The sensor arrangement may be configured to monitor air quality within the container. The control system may be configured to vary the pre-determined level of carbon dioxide based the air quality within the container.

Providing a detector that is able to vary the pre-determined level of carbon dioxide, above which an operator is altered, in relation to the air quality within the container has been found to improve the accuracy of determination of the occupancy of a container.

In order to monitor air quality, the first sensor may determine the temperature within the container, the level of humidity within the container, and/or the level of nitrogen within the container.

The mounting arrangement may comprise a first part for positioning outside of a container, and a second part for positioning within a container such that a section of a container is positioned therebetween. The mounting arrangement may further comprise at least one fastener configured and arranged to extend through an outer wall of a container, in use, for removably connecting the first and second parts together in order to mount the detector to a container.

In this way, the detector can easily be retro fitted to an existing container (e.g. by drilling suitable holes for the fastener to extend through. Moreover, providing at least a part of the mounting arrangement outside of the container enables an operator to easily inspect a container to determine whether or not it has been fitted with a detector.

The detector may comprise a first indicator for alerting an operator, wherein the control system output may be configured to activate the indicator when the concentration of carbon dioxide determined by the first sensor exceeds a predetermined value.

The first indicator may be configured and arranged to extend through an outer wall of a container, in use, such that at least a part of the first indicator is positioned outside of a container.

This arrangement ensures that at least a part of the first indicator is positioned outside of the container. This arrangement aids in the inspection of the occupancy of a container by making the first indicator visible outside of the container (useful for visual indicators) and by reducing any damping effects the container may have on an alarm (useful for an audio indicator).

The at least one fastener may be provided with a body in the form of a body a sleeve with a through bore, and wherein the first indicator extends through the through bore.

The indicator may comprise an audible indicator and/or a visual indicator.

The mounting arrangement may comprise a dampening arrangement for dampening vibrations between the detector and a container.

Mounting the detector a container via one or more dampeners reduces the shock/impact from the container to the detector, e.g. during movement/loading/emptying of the container. This reduces the impact damage imparted on the components of the detector, thus increasing the service life of the detector.

The dampening arrangement may comprise a first dampening member for positioning between the first part of the mounting arrangement and a container.

The dampening arrangement may comprise a second dampening member for positioning between the second part of the mounting arrangement and a container.

Providing first and second dampening members (e.g. shock absorbing washer) between both the mounting arrangement and the container has been found to effectively dampen the shock/impact/vibration between the container and the detector, e.g. during movement/loading/emptying of the container. This reduces the impact reduces damage imparted on the components of the detector.

The detector may comprise power storage unit disposed within the housing for providing power to the sensor arrangement and the alarm, wherein the power storage is mounted within the housing via an anti-vibration mounting arrangement.

Advantageously, this arrangement provides a detector as a self-contained unit that is able to be mounted within a container without the need for connection to an external power source. Mounting the power storage device to the housing reduces the shock/impact imparted from the container to the device (e.g. battery). This arrangement reduces the likelihood of the sensor or alarm becoming disconnected from the power device, which reduces the chances of failure of the detector.

The predetermined concentration of carbon dioxide may be in the range of 1000 ppm to 1500 ppm, preferably in the range of 1100 ppm to 1300 ppm, for example approximately 1200 ppm.

The approximate background level of carbon dioxide with a closed container is in the region of 400 ppm to 800 ppm. Detection of carbon dioxide levels in the ranges shown has been found to provide good sensitivity to occupancy of the container, whilst accommodating for variations in the background levels.

The first sensor may be configured to determine the composition of the air within the container in the region of every 20 to 40 minutes, preferably approximately every 30 minutes.

Carrying out readings of the air composition within this frequency range has been found to provide sufficiently regular safety checks, whilst maximising battery life.

The detector may comprise a transmitter for transmitting a signal indicative of the occupancy of a container to a processor at a remote location, when the sensor when the concentration of carbon dioxide sensed by the first sensor exceeds the predetermined value, and wherein the control system output is configured to activate the transmitter.

This arrangement advantageously allows for the occupancy of a container, or a series of containers, to be monitored remotely.

The detector may comprise a fire suppressant device disposed within the housing, and wherein the fire suppressant device may be configured to activate in response to detection of fire within the housing.

Use of a fire suppressant device, e.g. a fire suppressing aerosol, prevents fires from spreading from the detector to the container and causing damage to the container, or causing harm to a person within the container.

The sensor arrangement may comprise a second sensor configured to detect heat levels within a container.

The sensor arrangement may comprise a third sensor configured to detect motion within a container.

The housing may comprise a first chamber in which the first sensor is positioned and a second chamber in which the power storage device is positioned. The first chamber may be substantially sealed from the second chamber.

This arrangement seals the electronic components of the detector (e.g. the power storage device/battery) away from the first sensor/air flow path. This ensures that if water/debris enters the housing via the inlet or outlet, the electrical components are still protected.

According to a fourth aspect there is provided a container comprising: a body defining an internal space; and a detector according to the first aspect, the second aspect of the third aspect, said detector configured to monitor the internal space of the container.

The container may be a refuse container, a recycling container, a shipping container, or a trailer of a road vehicle.

### BRIEF DESCRIPTION OF THE DRAWINGS

Embodiments of the invention will now be described with reference to the accompanying drawings, in which:

FIG. 1 is a schematic view of a container including a detector according to an embodiment;

FIG. 2 is an exploded perspective view of a detector according to an embodiment;

FIG. 3 is a perspective view of the detector of FIG. 2 with the rear housing plate removed for clarity;

FIG. 4 is a rear view of the detector of FIG. 2 with the rear housing plate removed for clarity;

FIG. 5 is an exploded view of a detector according to an embodiment;

FIG. 6a is a rear perspective view of the detector of FIG. 5;

FIG. 6b is an enlarged partial view of the detector of FIG. 6a; and

FIG. 7 is a partial schematic side view of the detector of FIG. 5 mounted to a container.

### DETAILED DESCRIPTION OF EMBODIMENT(S)

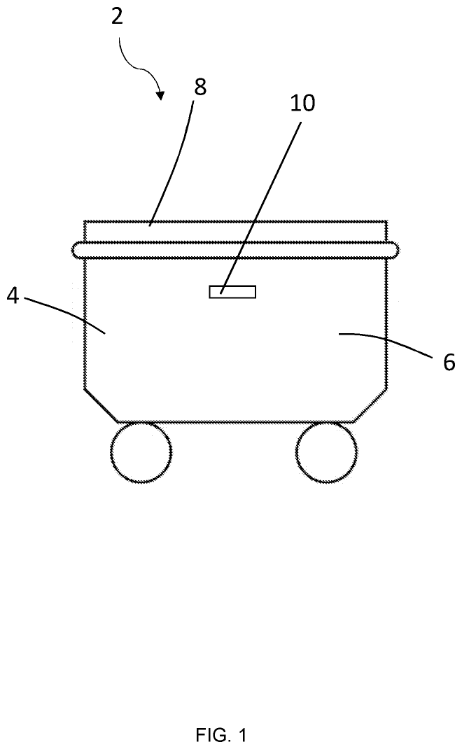

Referring to FIG. 1, a detector 10 is illustrated mounted to a container 2. The container 2 includes a body 4 defining an internal/enclosed space 6 and a lid 8 for opening and closing the container 2. The detector 10 is partially positioned within the internal space 6 of the container 2 and is configured to be able to determine the occupancy within the container 2 (i.e. the detector is configured to determine whether one or more people are in the container 2).

The detector 10 is configured to monitor the occupancy of the container 2 by sensing the composition of the air within the container 2. In the arrangement shown, the container body 4 includes four side walls, and the detector 10 is mounted to one of said side walls of the container 2. The detector 10 positioned a distance approximately a third of the height of the container, below the upper edge of the container. This positioning has been found to provide accurate measurements, and positions the detector 10 away from potential impacts with the lid 8. It will be appreciated that the detector 10 may be mounted to any surface of the container 2 so long as it is able to sense the composition of the air within the container 2. In alternative arrangements, the detector 10 may be mounted substantially centrally on the lid 8 of the container 2.

In the illustrated embodiment, the container 2 is shown as being an industrial refuse or recycling container. It will be appreciated that the detector 10 may be incorporated into any suitable container into order to determine the occupancy thereof, such as a shipping container or a trailer of a road vehicle.

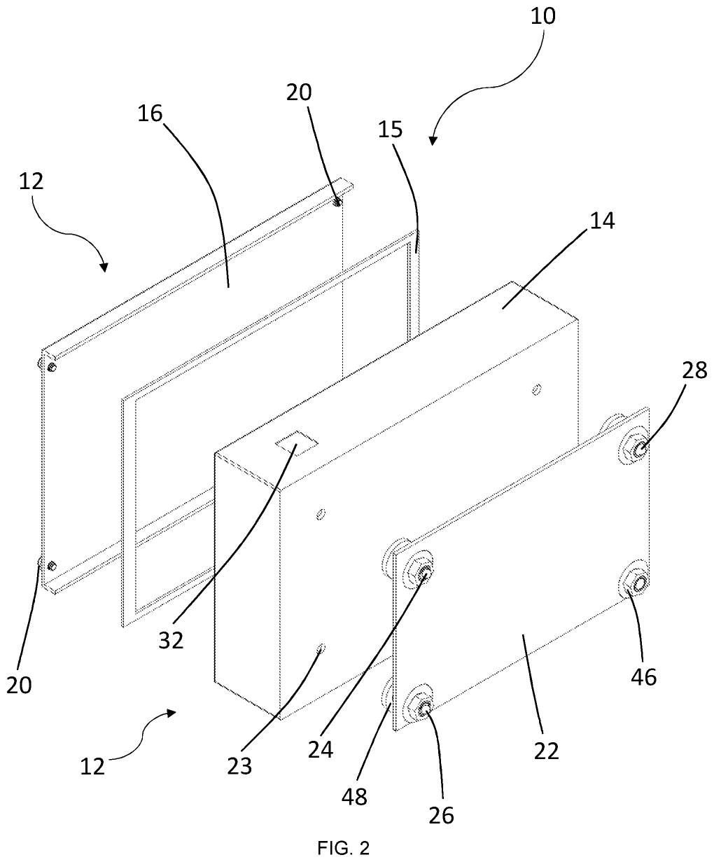

Referring now to FIG. 2, the detector 10 is illustrated in more detail. The detector 10 includes a housing 12 defining an internal volume. The housing 12 is provided with a first housing part 14 in the form of a main housing body 14 and a second housing part 16 in the form of a housing back plate 16. The housing body 14 is provided as an open topped box structure. The housing back plate 16 releasably securable to the housing body 14 via one or more fasteners 20. In the arrangement shown, the housing back plate 16 is secured to the housing body 14 via four fasteners 20, but any suitable number of fasteners may be used.

A gasket 15 is provided between the housing body 14 and the housing back plate 16 to provide a seal therebetween. Put another way, the housing back plate 16 releasably seals the internal cavity of the housing 12. In the illustrated embodiment, the housing 12 is substantially sealed so as to produce a dust-tight IP65 rated sealed housing. This arrangement prevents or restricts the ingress of dust into the housing 12 and minimises the ingress of water into the housing 12, which may damage the components of the detector 10.

A mounting arrangement is provided to secure the detector 10 to the container 2. The mounting arrangement includes a first part 22 positioned outside of the container 2. The first part is provided in the form a mounting plate 22. The mounting arrangement also includes a second part 23 positioned within the container 2. In the arrangement shown, the second part 23 is a surface of the housing 12 having apertures 23 therein. In this way, a section of the container 2 is positioned between the first part 22 and the second part 23 of the mounting arrangement.

The mounting arrangement includes fasteners (not shown) extending through an outer wall of the container 2 in order to secure the first and second parts 22, 23 of the mounting arrangement together, and so secure the detector 10 to the container 2.

In order to dampen any force transmitted between the container 2 and the detector 10 (e.g. when opening and closing the lid, when moving the container 2 and/or when loading/emptying the container 2), the mounting arrangement includes a dampening (e.g. vibration reducing) arrangement. Put another way, the mounting arrangement is configured to reduce the shock/impact imparted to the detector 10 from the container.

In the illustrated embodiment, the dampening arrangement includes a first dampening member 46 positioned between the first part 22 of the mounting arrangement and the container 2. The dampening arrangement includes a second dampening member 48 positioned between the second part 23 of the mounting arrangement and the container 2. In the arrangement shown, the first and second dampening members 46, 48 are provided as DIN 125A shock absorbing washers. It will be appreciated that any suitable arrangement for dampening forces imparted onto the detector 10 from the container 2 may be used.

The detector 10 also includes one or more indicators configured to alert an operator. In the arrangement shown, the indicators are be visual indicators that are arranged so as to be visible from outside of the container 2. Put another way, the indicators are at least partially positioned outside of the container 2. It will be appreciated that the indicators may also include audio indicator such as an alarm.

Each indicator is configured and arranged to extend through an outer wall of a container 2, in use, such that at least a part of each indicator is positioned outside of the container 2. A first indicator 24 is provided to alert an operator regarding the occupancy of the container 2. A second indicator 26 is provided to alert an operator regarding a fault with the detector 10. A third indicator 28 is provided to alert an operator to the detector 10 batteries running low. It will be appreciated that the first, second and/or third indicator 24, 26, 28 may include an audible indicator and/or a visual indicator.

As discussed above, the fasteners (not shown) of the mounting arrangement are configured and arranged to extend through an outer wall of the container 2 in order to secure the first and second parts 22, 23 of the mounting arrangement together. The fasteners are provided with a body in the form a sleeve having a through bore. Each indicator 24, 26, 28 is arranged to extend through the through bore so as to extend through an outer wall of the container 2. This allows the indicators 24, 26, 28 to be visible from outside of the container 2.

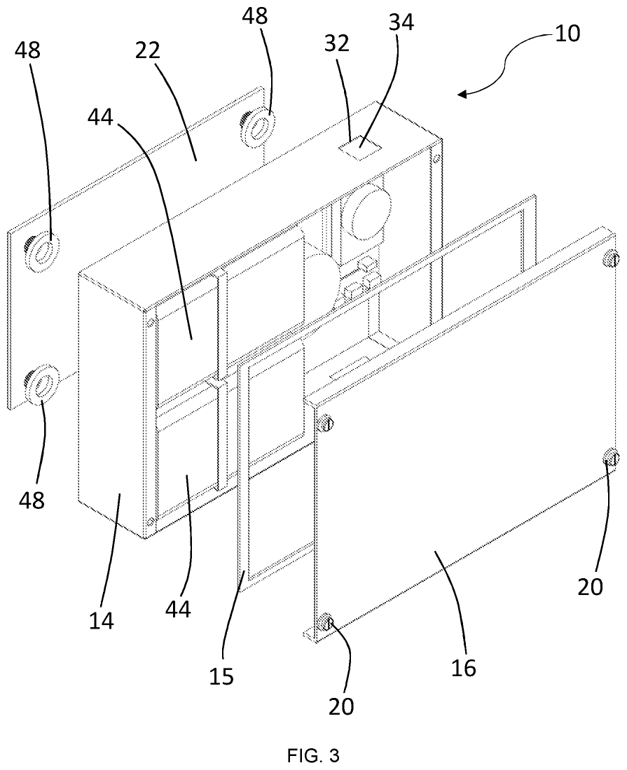

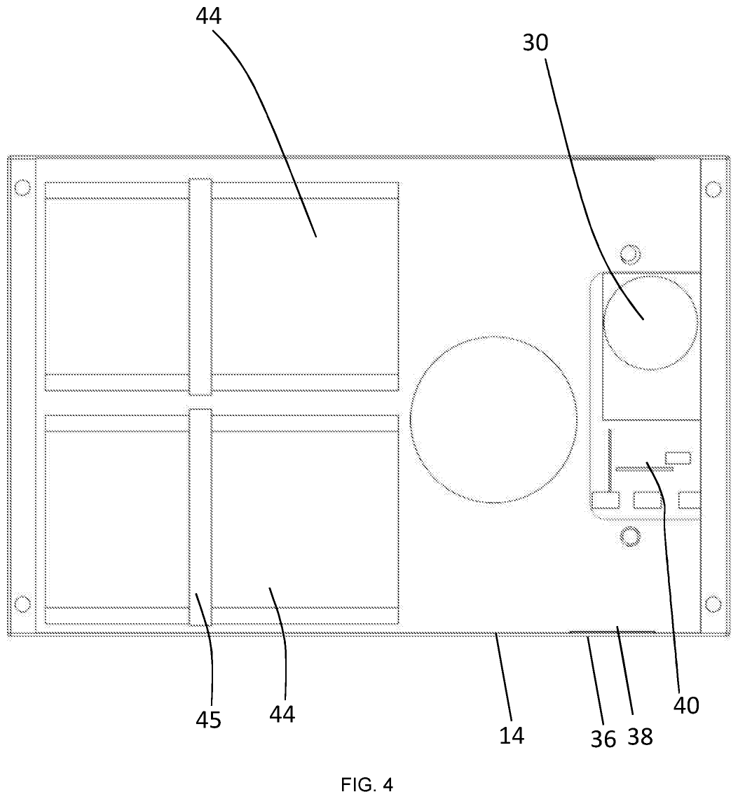

Referring now to FIGS. 3 and 4, the internal components of the detector 10 are illustrated.

The detector 10 includes a sensor arrangement disposed within the housing 12. The sensor arrangement includes a first sensor 30 for sensing the composition of the air within the container 2. The first sensor 30 is configured to sense and periodically monitor the concentration of different compounds within the container. In the present arrangement, the first sensor is configured to sense carbon dioxide in the air within the container 2. In some arrangements, the first sensor 30 may be provided as a volatile organic compound (VOC) sensor capable of detecting the presence and concentration of VOCs within the container 2.

In addition to carbon dioxide, the sensor arrangement may be configured to monitor air quality within the container. The first sensor 30 may be configured to monitor air quality. In order to monitor air quality, the first sensor 30 may determine the temperature within the container, the level of humidity within the container, and/or the level of nitrogen or VOCs within the container.

As discussed above, the detector 10 is configured to alert when the level of carbon dioxide within a container exceeds a pre-determined level. The detector 10 is able to vary the pre-determined level of carbon dioxide above which an indicator is activated, for example in response to the air quality detected within the container 2. In alternative arrangements, different sensors may be utilised for determining the concentration of carbon dioxide in the container 2, such as a nondispersive infrared (NDIR) gas sensor capable of detecting the presence and concentration of carbon dioxide within the container.

In order to prevent water from encountering the first sensor 30 (which may damage the sensor arrangement and affect the accuracy of the sensor readings), the first sensor 30 may be disposed within a watertight enclosure. As discussed above, the housing 12 is configured to be substantially sealed. The housing 12 is provided as a water-tight enclosure (e.g. IP65 rated). However, in alternative arrangements, it will be appreciated that the entire housing 12 may not be sealed so as to be water-tight. In some arrangements, only the sensor arrangement, e.g. the first sensor 30, may be provided within a water-tight enclosure.

The housing 12 is provided with an inlet 32 to enable air to flow into the housing 12 and over the first sensor 30, so that the air within the container 2 can be analysed by the first sensor 30. In the illustrated embodiment, the housing inlet 32 defines a diameter of approximately 12 mm, but may be in the range of 10 mm to 14 mm in order to provide sufficient air flow into the housing 12.

Due to the size of the inlet 32, the inlet 32 of the housing 12 is sealed with an inlet cover 34 substantially covering the inlet 32. The inlet cover 34 is configured to allow the passage of air therethrough and configured to prevent the flow of water therethrough. Put another way, the inlet cover 34 is provided as a semi-permeable membrane. The semi-permeable membrane is able to repel liquid water and dust, but allows air and water vapour to pass through into the housing 12. This arrangement provides an effective means of preventing liquid water/dust/debris ingress into the housing 12 whilst allowing air to flow into the housing 12 to be sensed. It will be appreciated that in alternative arrangements, the size of the inlet 32 may be reduced such that the inlet cover 34 is not required.

The housing 12 is provided with an outlet 36 to enable air to flow into the housing 12 and over the first sensor 30, so that the air within the container 2 can be analysed by the first sensor 30. In the illustrated embodiment, the housing outlet 36 defines a diameter of approximately 12 mm, but may be in the range of 10 mm to 14 mm in order to provide sufficient air flow into the housing 12.

The inlet 32 and the outlet 36 define an air flow path through the housing. Providing an air flow path through the housing 12 has been found to increase the air flow rate through the housing 12, and the first sensor 30 is disposed along the air flow path such that an increased amount of air flow flows over the first sensor 30.

The outlet 36 of the housing 12 is sealed with an outlet cover 38 substantially covering the outlet 36. The outlet cover 38 is configured to allow the passage of air therethrough and configured to prevent the flow of water therethrough. Put another way, the outlet cover 38 is provided as a semi-permeable membrane. The semi-permeable membrane is able to can repel liquid water and dust, but is able to allow air and water vapour to pass through into the housing 12. This arrangement provides an effective means of preventing water/dust/debris ingress into the housing 12 whilst allowing air to flow into the housing 12 to be sensed.

It will be appreciated that in alternative arrangements, the size of the inlet 32 may be reduced such that the inlet cover 34 is not required. In further alternative arrangements, it will be appreciated that the housing 12 may not be provided with a separate outlet 36, and the inlet 32 may function as the inlet and outlet.

In the arrangement shown, the inlet 32 is provided on a surface of the housing 12 that is intended to be lowermost in use, and the outlet 36 is provided on a surface of the housing 12 that is intended to be uppermost in use. Although not illustrated, the air flow path from the inlet 32 to the outlet 36 may be provided in the form of a duct (e.g. an enclosed duct) through the housing 12, where the first sensor 30 may be at least partially disposed within the duct. In addition to sealing the duct away from the other components of the detector 10, this arrangement may further increase air flow through the housing 12 via convection effects.

The detector 10 further includes a control system 40 configured to receive inputs values from the first sensor 30, and to provide an output when the concentration of carbon dioxide exceeds a predetermined value.

The predetermined value for the concentration of carbon dioxide may typically by in the range of 1000 ppm to 1500 ppm, or in the range of 1100 ppm to 1300 ppm. In the illustrated embodiment, the predetermined value for the concentration of carbon dioxide is approximately 1200 ppm. The approximate background level of carbon dioxide with a closed container is in the region of 400 ppm to 800 ppm. Detection of carbon dioxide levels in the ranges shown has been found to provide good sensitivity to occupancy of the container, whilst accommodating for variations in the background levels.

In some embodiments, the controller 40 is configured to provide an output when the level of humidity within the container 2 exceeds a predetermined value, when the temperature within the container 2 exceeds a predetermined threshold and/or when the concentration of VOCs exceeds a predetermined value.

In order to provide power to the sensor arrangement and the indicators, the detector 10 includes a power storage unit 42, e.g. one or more batteries, disposed within the housing 12. The enables the detector 10 to be a self-contained unit, without the need for connection to an external power source.

Although not illustrated, the housing 12 may include a first chamber in which the first sensor 30 is positioned and a second chamber in which the power storage device is positioned, and wherein first chamber is substantially sealed from the second chamber. This arrangement seals the electronic components of the detector (e.g. the power storage device/battery) away from the first sensor/air flow path. This ensures that if water/debris enters the housing via the inlet or outlet, the electrical components are still protected. In alternative arrangements, power storage unit 42 may be provided within a water-tight enclosure within the housing 12.

The power storage unit 42 includes two batteries 44. Each of the batteries 44 is mounted to the housing 12 via an anti-vibration mounting arrangement. The anti-vibration mounting arrangement is provided in the form of a mounting bracket 45 secured to the housing via one or more shock absorbing washers (not shown). This arrangement works to dampen the shock/impact imparted from the container 2 to the power storage unit 42 (e.g. batteries 44). It will be appreciated that the number of batteries provided will vary to suit the application.

Although not illustrated, in alternative arrangements the detector 10 may also include a transmitter for transmitting a signal indicative of the occupancy of a container to a processor at a remote location, when the sensor when the concentration of carbon dioxide sensed by the first sensor exceeds the predetermined value. This arrangement advantageously allows for the occupancy of a container, or a series of containers, to be monitored remotely.

The sensor arrangement may also include a sensor configured to detect the temperature within the container 2. The controller 40 may be configured to provide an output when the temperature within the container 2 exceeds a predetermined value. This helps to improve the accuracy of occupancy determination for a container, as this increase in temperature within a container may be due to a human occupant's body heat.

In some arrangements, the sensor arrangement may be configured to detect the temperature outside of a container 2. In such embodiments, the control system 40 may be configured to provide an output when the difference between the temperature within the container and outside of the container exceeds a predetermined value. This improves the accuracy of occupancy determination based on temperature within a container regardless of the ambient temperature outside of the container.

The sensor arrangement may include a sensor configured to detect motion within a container 2. In such embodiments, the control system 40 may be configured to provide an output when motion within the container 2 has been detected. This helps to further increase the accuracy of the occupancy detection.

The sensor arrangement may also include a sensor configured to determine an orientation of a container 2. For example, the fourth sensor may measure or estimate a change in angle of one or more axes of a container 2 with respect to a reference axis, such as gravity. The fourth sensor may be, for example, a gyroscope and/or an accelerometer. In such arrangements, the sensor arrangement may be configured to monitor when the container 2 is emptied and/or filled. In arrangements where the detector 10 is mounted directly onto the lid 8 of the container 2, this arrangement would also keep a log of when the lid 2 was opened. This in turn would enable the determination of whether the container 2 had been opened by an authorised person.

The detector 10 may be provided with a fire suppressant device. The fire suppressant device may be disposed within the housing 12. The detector 10 may be configured to activate the fire suppressant device in response to detection of fire within the housing 12, e.g. through the detection of smoke within the housing 12.

The detector 10 may be provided with a positioning system (e.g. it could be fitted with a GPS tracker) to enable the location of the container 2 to be tracked. In this way, should it be determined that there is an occupant of a container 2, it would be possible to remotely determine where the occupant was located.

The control system may be configured to calculate the average values for each sensor over a pre-determined period of time. This would enable the control system to determine a mean value for each sensor for a given time frame. Put another way, the control system 40 may comprise a memory and a processor configured to execute an algorithm. The algorithm may be configured to store input values received by the control system 40 from the sensors in the sensor arrangement and store them in the memory to determine the average value thereof. Significant variations the calculated average values may cause the control system 40 to provide an output. These calculated average values help to increase the accuracy of the occupancy determination, and the control system 40 is able to determine base average values for a particular location/time of year.

In some arrangements, the sensor arrangement may incorporate a cardioballistic detector configured to detect a heartbeat of an occupant within a container. In such arrangements, the control system 40 may be configured to provide an output when the cardioballistic detector detects a heartbeat within a container.

It will be appreciated that in arrangements of the detector 10 incorporating two or more of the sensors discussed, the control system 40 may be configured to only provide an output when multiple sensors, e.g. all of the sensors, indicate that the container is occupied.

Put another way, the control system 40 may be configured to only provide an output when several or all of the following occurs: the concentration of carbon dioxide exceeds a predetermined value; the temperature within a container exceeds a predetermined value or the temperature differential between inside and outside of the container exceeds a predetermine value; motion is detected within a container; concentration of VOCs exceeds a predetermined value; and/or when a cardioballistic detector detects a heartbeat within a container.

The control system 40 may provide such an output when one condition is met (e.g. if one monitored value exceeds a predetermined amount) or may provide an output when two or more conditions are met. For example, the control system 40 may only provide such an output when the concentration of carbon dioxide within the container 2, as determined by the first sensor 30, exceeds a predetermined value and when motion has been detected within the container 2 as determined by the fourth sensor. This may reduce the likelihood of false alarm events, thus increasing the accuracy of the occupancy determination.

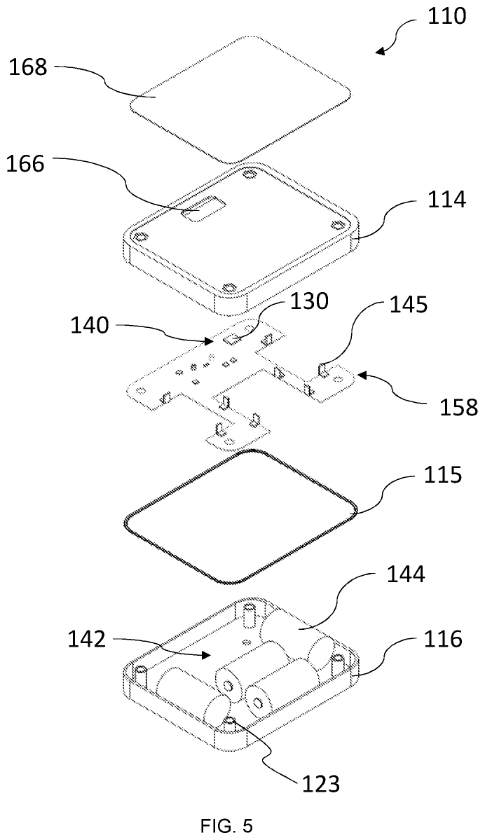

Referring now to FIG. 5, a detector is illustrated and indicated generally at 110. Similar features of the detector 110 with respect to the detector 10 of FIGS. 1 to 4 are labelled with the prefix “1”.

The detector 110 includes a housing 112 defining an internal volume. The housing 112 is formed from a first housing part 114 releasably secured to a second housing part 116. In the present arrangement the housing parts 114, 116 are of substantially the same shape, both having a generally rectangular cross-sectional profile. In alternative embodiments the housing 112 may be formed from housing parts 114, 116 having a different cross-sectional shape, such as circular housing parts for example. Further, in some arrangements the housing 112 may be provided as an open top box structure and a plate so as to close/seal the open top box.

The housing parts 114, 116 are releasably secured together via four fasteners (not shown). Each fastener extends through openings in both the first housing part 114 and the second housing part 116. The openings for the fasteners are located in the each of the four corners of the housing parts 114, 116, but it will be appreciated that any suitable number of fasteners and locations may be used.

A gasket 115 is provided between the two housing parts 114, 116, to provide a seal therebetween. The gasket 115 ensures that the housing 112 is substantially sealed. It will be appreciated that the housing 112 may be sealed so as to produce a dust-tight IP65 rated sealed housing 112. This helps to prevent or restrict the ingress of dust into the housing 112 and minimises the ingress of water into the housing 112, which may damage the components of the detector 110.

The detector 110 includes a sensor arrangement disposed within the housing 112. The sensor arrangement includes a first sensor 130 for sensing the composition of the air within the container 102. The first sensor 130 is configured to sense and periodically monitor the concentration of different compounds within the container 102. In the present arrangement, the first sensor 130 is configured to sense carbon dioxide in the air within the container 102. In some arrangements, the first sensor 130 may be provided as a volatile organic compound (VOC) sensor capable of detecting the presence and concentration of VOCs within the container 102.

In addition to carbon dioxide, the sensor arrangement may be configured to monitor air quality within the container. The first sensor 130 may be configured to monitor air quality. In order to monitor air quality, the first sensor 130 may determine the temperature within the container, the level of humidity within the container, and/or the level of nitrogen or VOCs within the container.

The detector 110 further includes a control system 140 configured to receive inputs values from the first sensor 130, and to provide an output when the concentration of carbon dioxide exceeds a predetermined value. The detector 110 is able to vary the pre-determined level of carbon dioxide above which an indicator is activated, for example in response to the air quality detected within the container 102. In alternative arrangements, different sensors may be utilised for determining the concentration of carbon dioxide in the container 102, such as a nondispersive infrared (NDIR) gas sensor capable of detecting the presence and concentration of carbon dioxide within the container.

The predetermined value for the concentration of carbon dioxide may typically by in the range of 1000 ppm to 1500 ppm, or in the range of 1100 ppm to 1300 ppm. In the illustrated embodiment, the predetermined value for the concentration of carbon dioxide is approximately 1200 ppm. The approximate background level of carbon dioxide with a closed container is in the region of 400 ppm to 800 ppm. Detection of carbon dioxide levels in the ranges shown has been found to provide good sensitivity to occupancy of the container, whilst accommodating for variations in the background levels.

In the embodiment illustrated a circuit board 158 is located within the housing 112. The circuit board 158 includes the control system 140 and sensor arrangement for determining the occupancy of the container 102.

The detector 110 also includes one or more indicators configured to alert an operator. Each indicator is configured and arranged to extend through an outer wall of a container 102, in use, such that at least a part of each indicator is positioned outside of the container 102. A first indicator 124 is provided to alert an operator regarding the occupancy of the container 102. A second indicator 126 is provided to alert an operator regarding a fault with the detector 10. A third indicator 128 is provided to alert an operator to the detector 110 batteries running low. It will be appreciated that the first, second and/or third indicator 124, 126, 128 may include an audible indicator and/or a visual indicator.

The indicator(s) are visible from outside of the housing 112 via a cut-out 166 in the first housing part 114 through a transparent or translucent cover 168. The cover 168 is secured to an external face of the first housing part 114. The cover 168 also works to substantially seal the housing 112 to prevent the ingress of water and/or debris.

In order to provide for providing power to the sensor arrangement and the indicators, the detector 110 includes a power storage unit 142 disposed within the housing 112. The enables the detector 110 to be a self-contained unit, without the need for connection to an external power source.

The power storage unit 142 includes four batteries located within the internal volume defined by the housing 112. Each of the batteries is mounted to the housing 112 via an anti-vibration mounting arrangement. The anti-vibration mounting arrangement is provided in the form of a mounting bracket 145 secured to the housing via one or more shock absorbing washers (not shown). This arrangement works to dampen the shock/impact imparted from the container 2 to the power storage unit 142 (e.g. batteries 144). It will be appreciated that the number of batteries provided will vary to suit the application. In the arrangement shown, the mounting brackets 145 are provided in the form of a clip.

Although not illustrated, in alternative arrangements the detector 10 may also include a transmitter for transmitting a signal indicative of the occupancy of a container to a processor at a remote location, when the sensor when the concentration of carbon dioxide sensed by the first sensor exceeds the predetermined value. This arrangement advantageously allows for the occupancy of a container, or a series of containers, to be monitored remotely.

The sensor arrangement includes a sensor configured to detect the humidity within the container 102. The controller 140 may be configured to provide an output when the level of humidity within the container 102 exceeds a predetermined value. This helps to improve the accuracy of occupancy determination for a container, as this increase in temperature within a container may be due to a human occupant's body heat.

The sensor arrangement includes a sensor configured to detect the temperature within the container 102. The controller 140 provides an output when the temperature within the container 102 exceeds a predetermined value. This helps to improve the accuracy of occupancy determination for a container, as this increase in temperature within a container may be due to a human occupant's body heat. The sensor arrangement further includes a sensor configured to detect the temperature outside of a container 102. The control system 140 is configured to provide an output when the difference between the temperature within the container 102 and outside of the container 102 exceeds a predetermined value. This improves the accuracy of occupancy determination based on temperature within a container, as it is able to take seasonal variations in temperature or different temperatures at different locations into account.

The sensor arrangement includes a sensor configured to detect motion within the container 102. In such embodiments, the control system 140 provides an output when motion within the container 102 has been detected. This helps to further increase the accuracy of the occupancy detection.

In some embodiments, the controller 140 is configured to provide an output when the level of humidity within the container 102 exceeds a predetermined value, when the temperature within the container 102 exceeds a predetermined threshold, when the concentration of VOCs exceeds a predetermined value, and/or when motion is detected within the container 102.

The sensor arrangement includes a sensor configured to determine an orientation of a container 102. Put another way, the sensor arrangement includes a sensor to monitor a change in angle of one or more axes of a container 102 with respect to a reference axis, such as gravity. The sensor may be, for example, a gyroscope and/or an accelerometer. In such arrangements, the sensor arrangement may be configured to monitor when the container 102 is emptied and/or filled. In arrangements where the detector 110 is mounted directly onto the lid 108 of the container 102, this arrangement would also keep a log of when the lid 102 was opened. This in turn would enable the determination of whether the container 102 had been opened by an authorised person.

The detector 110 is configured to change the first indicator 124 from an active state, in which the indicator 124 alerts an operator regarding the occupancy of the container 102, to an inactive state. The detector 110 is configured such that this change from active to inactive state occurs when the container 102 has been rotated by predetermined amount, e.g. when it has been rotated by angle of approximately 90 degrees. This provides a mechanism by which the first indicator 124 may be reset by the action of emptying the container 102, for example, emptying the contents of the container 102 into a bin lorry.

The detector 110 may be provided with a fire suppressant device. The fire suppressant device may be disposed within the housing 112. The detector 110 may be configured to activate the fire suppressant device in response to detection of fire within the housing 112, e.g. through the detection of smoke within the housing 112.

The detector 110 may be provided with a positioning system (e.g. it could be fitted with a GPS tracker) to enable the location of the container 102 to be tracked. In this way, should it be determined that there is an occupant of a container 102, it would be possible to remotely determine where the occupant was located.

As discussed above, the detector 110 includes a control system 140. The control system 140 may be configured to calculate the average values for each sensor over a pre-determined period of time. This would enable the control system to determine a mean value for each sensor for a given time frame. Put another way, the control system 140 may comprise a memory and a processor configured to execute an algorithm. The algorithm may be configured to store input values received by the control system 140 from the sensors in the sensor arrangement and store them in the memory to determine the average value thereof. The readings may be continuously taken and recorded and the average/mean value continuously updated.

Significant variations the calculated average values may cause the control system 140 to provide an output. These calculated average values help to increase the accuracy of the occupancy determination, and the control system 140 is able to determine base average values for a particular location/time of year. Through this determination of the average or mean values for each of the monitored variables, the detector is able to determine the occupancy of a container 102 based on the base values for a given location of a container 102. This helps to increase the accuracy of the occupancy detection for a container 102.

In some arrangements, the sensor arrangement may incorporate a cardioballistic detector configured to detect a heartbeat of an occupant within a container 102. In such arrangements, the control system 140 may be configured to provide an output when the cardioballistic detector detects a heartbeat within a container.

It will be appreciated that in arrangements of the detector 110 incorporating two or more of the sensors discussed, the control system 140 may be configured to only provide an output when multiple sensors, e.g. all of the sensors, indicate that the container is occupied.

Put another way, the control system 140 may be configured to only provide an output when several or all of the following occurs: the concentration of carbon dioxide exceeds a predetermined value; the temperature within a container exceeds a predetermined value or the temperature differential between inside and outside of the container exceeds a predetermine value; motion is detected within a container; concentration of VOCs exceeds a predetermined value; and/or when a cardioballistic detector detects a heartbeat within a container.

It will be appreciated that the sensor arrangement is configured to monitor the conditions within the container regularly. For example the sensor arrangement may be configured to monitor the container at least every minute, optionally every 30 seconds, for example every 10 seconds.

The control system 140 may provide such an output when one condition is met (e.g. if one monitored value exceeds a predetermined amount) or may provide an output when two or more conditions are met. For example, the control system 140 may only provide such an output when the concentration of carbon dioxide within the container 102, as determined by the first sensor 130, exceeds a predetermined value and when motion has been detected within the container 102 as determined by the fourth sensor. This may reduce the likelihood of false alarm events, thus increasing the accuracy of the occupancy determination.

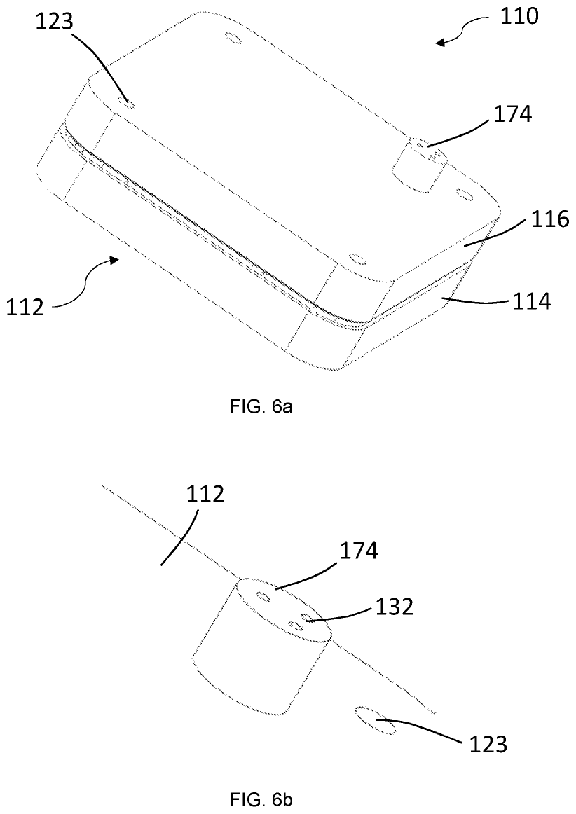

As is illustrated in FIGS. 6a and 6b, the housing 112 includes a sensor housing portion 174 extending from the housing 112. As best seen in FIG. 6b, the sensor housing portion 174 defines an inlet 132 that allows air to flow into the sensor housing portion 174. The inlet 132 is provided in the form of three apertures, but it will be appreciated that any suitable number of apertures may be used. Each aperture of the inlet 132 has a diameter in the range 0.5 mm to 4 mm. Often, each aperture 132 may have a diameter in the range 1 mm to 3 mm, for example the diameter of the inlet may be approximately 2 mm. It will be appreciated that the apertures of the inlet 132 may be dimensioned so as to seal the housing 112 from the ingress of water and/or debris. In the illustrated arrangement, the inlet 132 includes three apertures, but it will be appreciated that the number of apertures may be vary.

The sensor housing portion 174 has a circular cylindrical shape. However, it will be appreciated that in alternative embodiments (not illustrated), the sensor housing portion 174 may be cuboidal, frustoconical or any suitable three-dimensional shape.

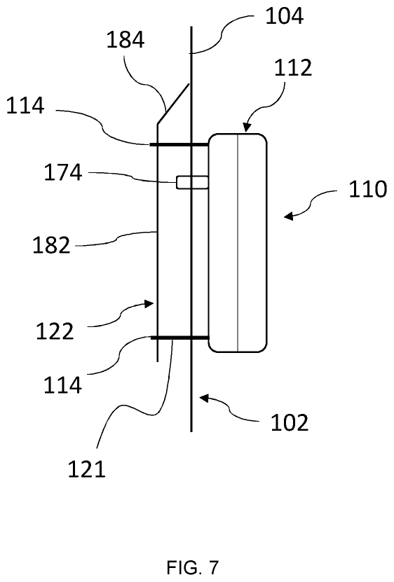

The sensor housing portion 174 is configured and arranged to extend through an opening in an outer wall of the container 102 when the detector 110 is mounted to the container 102 (see FIG. 7). The sensor housing portion 174 is at least partially positioned within the container 102. The present mounting arrangement of the detector increases the visibility of the detector 110 whilst enabling the detector 110 to monitor the conditions within the container 102. It will be appreciated that in the some arrangements the sensor housing portion 174 may be substantially flush with an inner surface of the wall 104 of the container 102.

In the illustrated arrangement, the inlet 132 is not covered. However, in some alternative arrangements, e.g. when the apertures of the inlet 132 are larger, the housing 112 may be provided with a cover substantially covering the inlet 132. The inlet cover may be configured to allow the passage of air therethrough and configured to prevent the flow of water therethrough. Put another way, the cover may be provided as a semi-permeable membrane configured to allow the passage of air therethrough and configured to prevent the passage of water therethrough. As illustrated in FIG. 7, a mounting arrangement is provided to secure the detector 110 to the container 102. The mounting arrangement includes a first part 122 positioned within a container 102. The first part is provided in the form a mounting plate 122. The mounting arrangement also includes a second part 123 positioned outside the container 102. In the arrangement shown, the second part 123 is a surface of the housing 112 having apertures 123 therein.

In the embodiment illustrated, the second housing part 116 of the detector 110 is configured to be mounted to an external wall of a container 2. This improves visibility of the detector 110 and positions the indicators such that they face outwardly from the container 102. Through this positioning, an operator is able to quickly determine that detector 110 has been fitted to the container 102, and to determine the occupancy thereof.

The mounting arrangement includes fasteners 121 extending through an outer wall 104 of the container 102 in order to mount the detector 110 to the container 102. The fasteners 121 secure the first and second parts 122, 123 of the mounting arrangement together, so as to secure the detector 110 to the container 102. The detector 110 is mounted substantially centrally on the container 102. In the arrangement shown, the mounting arrangement for securing the detector 110 to a container 102 is the same as the fastening arrangement for securing the first and second housing parts 114, 116 together. It will be appreciated that in alternative arrangements the mounting arrangement a fastening arrangement may be provided separately.

The mounting plate 122 is positioned within the internal space of the container 102. The mounting plate 122 is secured to the housing 112 via the fasteners extending through openings in the container 102 to secure the detector 110 to the container 102. As such, a section of the container 2 is positioned between the mounting plate 122 and the housing 112. In this way, a section of the container 102 is positioned between the first part 122 and the second part 123 of the mounting arrangement.

The mounting plate 122 is positioned so as to substantially cover the sensor housing portion 174 (and so the inlet 132) from the impacts with contents of the container 102.

The mounting plate 122 includes an angled upper region 184. The upper region 184 of the mounting plate 122 is angled towards the wall 104 of the container 102 (i.e. towards the housing 112). The angled upper region 184 is arranged so as to be positioned above the sensor housing portion 174 (and so the inlet 132) so as to protect the inlet 132 from objects being placed or dropped into the container 102 from above.

In order to dampen any force transmitted between the container 102 and the detector 110 (e.g. when opening and closing the lid, when moving the container 102 and/or when loading/emptying the container 102), the mounting arrangement includes a dampening (e.g. vibration reducing) mounting arrangement. Put another way, the mounting arrangement 118 is configured to reduce the shock/impact imparted to the detector 110 from the container 102.

In the illustrated embodiment, the dampening arrangement includes dampening members provided as DIN 125A shock absorbing washers. It will be appreciated that any suitable arrangement for dampening forces imparted onto the detector 110 from the container 102 may be used.

Although the invention has been described above with reference to one or more preferred embodiments, it will be appreciated that various changes or modifications may be made without departing from the scope of the invention as defined in the appended claims.

1. A detector for mounting to a container defining an internal space to determine occupancy of the container, the detector comprising:
a housing defining an internal volume;
a mounting arrangement for mounting the housing to a container; a sensor arrangement comprising a first sensor disposed within the housing, the first sensor configured to determine a concentration of carbon dioxide within an internal space of a container; and
a control system configured to receive input values from the first sensor and to provide an output when the concentration of carbon dioxide exceeds a predetermined value,
wherein the housing is substantially sealed for restricting the ingress of debris into the housing, and
wherein the housing comprises an inlet such that air is able to flow into the housing and over the first sensor.

2. A detector according to claim 1, wherein the detector is configured to be mounted to an external surface of a container, in use, and wherein the housing comprises a sensor housing portion defining the inlet for air to flow into the sensor housing portion, the sensor housing portion projecting from the housing and configured and arranged to extend into a container, in use, through an opening of a wall of a container.

3. (canceled)

4. A detector according to claim 1, wherein the inlet has a diameter in the range of 1 mm to 3 mm, for example the diameter of the inlet is approximately 2 mm.

5. A detector according to claim 1, wherein the housing comprises a cover substantially covering the inlet, and wherein the cover is provided as a semi-permeable membrane configured to allow the passage of air therethrough and configured to prevent the passage of water therethrough.

6. (canceled)

7. A detector according to claim 1, wherein the mounting arrangement comprises a mounting plate for positioning within a container such that a section of a container is positioned between the mounting plate and the housing, in use.

8. (canceled)

9. A detector according to claim 1, wherein the mounting arrangement comprises a dampening arrangement configured to dampen, vibrations between the detector and a container, in use, optionally wherein the dampening arrangement comprises a first dampening member for positioning between the housing and the container, in use.

10. A detector according to claim 1, comprising a first indicator configured to indicate whether or not a container is occupied based on the determined container occupancy, optionally wherein the indicator comprises an audible indicator and/or a visual indicator.

11. A detector according to claim 1, wherein the output of the control system changes a first indicator from an interactive state to an active state to alert an operator that a container is occupied, in use, and wherein the detector is configured to change the first indicator from an active state to an inactive state during unloading of a container, in use.

12. A detector according to claim 11, wherein the sensor arrangement comprises a sensor configured to determine an orientation of a container, in use, and wherein the control system is configured to change the first indicator from an active state to an inactive state when the container has been rotated by at least a predetermined angle, optionally wherein the predetermined angle is approximately 90 degrees.

13. A detector according to claim 1, wherein the sensor arrangement is configured to detect the temperature within a container, and wherein the control system is configured to receive input values from the sensor arrangement and to provide an output when the temperature within the container exceeds a pre-determined value.

14. A detector according to claim 13, wherein the sensor arrangement is configured to detect temperature outside of a container, and wherein the control system is configured to receive input values from the sensor arrangement and to provide an output when the difference between the temperature inside a container and the temperature outside of a container exceeds a pre-determined value.

15. A detector according to claim 1, wherein the sensor arrangement is configured to detect motion within a container, and wherein the control system is configured to receive input values from the sensor arrangement and to provide an output when motion within a container is detected, in use.

16. A detector according to claim 1, wherein the sensor arrangement is configured to monitor humidity within a container, and wherein the control system is configured to receive input values from the sensor arrangement and to provide an output when the humidity within a container exceeds a predetermined value.

17. A detector according to claim 1, wherein the first sensor is configured to determine a concentration of volatile organic compounds (VOCs) within the internal space of the container, and optionally, wherein the control system is configured to provide an output when the concentration of VOCs exceeds a predetermined value.

18. A detector according to claim 1, wherein the control system is configured to determine a mean value from the input values received from the sensor arrangement, and wherein the control system is configured to provide an output when an input value differs from its associated mean value by a predetermined amount.

19. (canceled)

20. A detector according to claim 1, comprising a power storage unit disposed within the housing for providing power to the sensor arrangement and the alarm, wherein the power storage is mounted within the housing via an anti-vibration mounting arrangement.

21. A detector according to claim 1, wherein the predetermined concentration of carbon dioxide is in the range of 1000 ppm to 1500 ppm, preferably in the range of 1100 ppm to 1300 ppm, for example approximately 1200 ppm.

22-25. (canceled)

26. A detector according to claim 1, wherein the housing comprises first and second housing parts that are releasably secured together, and comprises a gasket positioned between the first and second housing parts so as to seal the housing.

27. A detector for mounting to a container defining an internal space to determine occupancy of the container, the detector comprising:
a housing defining an internal volume;
a mounting arrangement for mounting the housing to a container; a sensor arrangement comprising a first sensor disposed within the housing, the first sensor configured to determine a concentration of carbon dioxide within an internal space of a container; and
a control system configured to receive input values from the first sensor and to provide an output when the concentration of carbon dioxide exceeds a predetermined value,
wherein the housing is substantially sealed for restricting the ingress of debris into the housing, and
wherein the housing comprises an inlet such that air is able to flow into the housing and over the first sensor,
wherein the inlet has a diameter in the range of 1 mm to 3 mm and/or wherein the housing comprises a cover covering the inlet in the form of a semi-permeable membrane configured to allow the passage of air therethrough and configured to prevent the passage of water therethrough.

28. A detector for mounting to a container defining an internal space to determine occupancy of the container, the detector comprising:
a housing defining an internal volume;
a mounting arrangement for mounting the housing to a container; a sensor arrangement comprising a first sensor disposed within the housing, the first sensor configured to determine a concentration of carbon dioxide within an internal space of a container;
a power storage unit disposed within the housing for providing power to the detector; and
a control system configured to receive input values from the first sensor and to provide an output when the concentration of carbon dioxide exceeds a predetermined value,
wherein the housing is substantially sealed for restricting the ingress of debris into the housing,
wherein the housing comprises an inlet such that air is able to flow into the housing and over the first sensor, and
wherein the detector comprises an anti-vibration mounting arrangement configured to dampen vibrations between the housing and the power storage unit and/or to dampen vibrations between the detector and a container, in use.
